(19) United States
(12) Patent Application Publication
(10) Pub. No.: US 2003/0108621 A1
(43) Pub. Date: Jun. 12, 2003

(54) Use of iodide salt as an aphrodisiac

(75) Inventor: Susann Reilly, Washington, DC (US)

Correspondence Address: FOLEY AND LARDNER, SUITE 500, 3000 K STREET NW, WASHINGTON, DC, 20007, US

(73) Assignee: RAINBURST, LLC

(21) Appl. No.: 10/166,741

(22) Filed: Jun. 12, 2002

Related U.S. Application Data
(63) Continuation of application No. 09/984,007, filed on Oct. 26, 2001, now abandoned.
(60) Provisional application No. 60/243,402, filed on Oct. 27, 2000.

(51) Int. Cl. A61K 33/42; A61K 33/36; A61K 31/19; A61K 35/78

(52) U.S. Cl. 424/669; 424/605; 424/736; 514/557; 514/574

(57) ABSTRACT

A method of increasing vaginal lubrication in an adult human female by orally administering to the female an effective amount of an aqueous solution having a pharmaceutically acceptable water-soluble iodide salt.

### BACKGROUND OF THE INVENTION

The normal sexual response begins with sexual arousal which causes genital vasocongestion that results in vaginal lubrication. The lubrication is due to the formation of a transudate in the vagina which, in conjunction with genital congestion, produces the so-called orgasmic platform prior to orgasm. Sexual stimuli and healthy vaginal tissue are prerequisites for genital vasocongestion and vaginal lubrication, (HARRISON'S PRINCIPLES OF INTERNAL MEDICINE, 12th ed., ed Wilson, J. D., et al. p. 301, McGraw-Hill Inc., New York (1991)).

Illnesses that impair neurological function, such as diabetes mellitus, may prevent normal sexual arousal. Pelvic diseases, such as vaginitis or endometriosis may also interfere with the normal sexual response. More commonly, decreased sexual response is due to psychological factors such as feelings of guilt, stresses such as anxiety, depression, fatigue or interpersonal conflicts which lead to failure of the vasocongestive response and prevent sufficient vaginal lubrication. (HARRISON'S PRINCIPLES OF INTERNAL MEDICINE, 12th ed., ed. Wilson, J. D., et al. p. 301, McGraw-Hill Inc., New York (1991)). In addition, vaginal lubrication in response to sexual stimuli may decrease as a woman ages. Insufficient, or absence of, vaginal lubrication may result in unsatisfying, or painful sexual relations.

Women experiencing stress may seek professional counseling by a therapist, physician or psychiatrist to overcome such problems and achieve normal sexual response. However, this option is often costly and time-consuming. Women suffering from a physical illness may be able to obtain relief through treatment of the underlying physical illness.

Alternatively, a number of creams or ointment preparations are commercially available for artificial lubrication. However, these preparations are often messy and inconvenient to use. Moreover, physical application of these artificial preparations are not always hygienic, and may introduce contaminants to the vaginal area, leading to irritation or infection. Thus, the need exists for a neat, hygienic, and convenient method of stimulating vaginal lubrication.

### SUMMARY OF THE INVENTION

This invention relates to a method of increasing, or stimulating vaginal lubrication in an adult human female by generating, in situ, hydriodic acid by administering an aqueous solution of iodide salt or a solution of an iodide salt and an acid. In the method of the present invention, the iodide salt is administered orally in an aqueous solution that may further comprise a pharmaceutically acceptable inorganic or organic acid. Approximately 24 hours after oral administration, the amount of transudate present in the vagina is increased, resulting in increased lubrication of the vagina.

Oral administration of iodide salt to increase vaginal lubrication eliminates the problems associated with the application of creams and ointments to the vaginal area, thus simplifying preparation for sexual encounters and enhancing sexual satisfaction. Moreover, the risk of infection associated with external application of artificial lubricants is greatly reduced if not all together eliminated with the present invention.

### DETAILED DESCRIPTION OF THE INVENTION

This invention is based upon the discovery that an aqueous solution of iodide salt can heighten sexual desire, including increasing the amount of transudate present in the vagina, resulting in increased vaginal lubrication.

Hydriodic acid, which is generated in situ by the present invention, is a clear colorless, or pale yellow liquid, an aqueous solution of hydrogen iodide, which is a gas at ordinary temperature. Hydriodic acid is also known as anhydrous hydriodic acid, hydrogen iodide (HI), hydrogen monoiodide or hydriodic. (The Merck Index, Merck & Co. Inc., 10th Ed. (1983)). Typically, hydriodic acid has been used as an expectorant, especially in the treatment of whooping cough (Pertussis).

It has been known from U.S. Reissue Pat. No. 59,1352, which reissued from U.S. Pat. No. 5,281,423, that an aqueous solution of hydriodic acid has aphrodisiac properties, as confirmed through its use by an adult human female. The hydriodic acid of that invention consists of hydriodic acid syrup comprised of hydriodic acid, water and dextrose. In that reissue, hydriodic acid syrup was orally administered (ingested) to the subject. After approximately 24 hours, an aphrodisiac effect was experienced by the subject. Surprisingly, the inventor has discovered that the same effect may be achieved by orally administering an iodide salt.

The terms “aphrodisiac” or “heightened sexual desire,” for the purposes of the present invention, refer to the following physical manifestations including clitoral swelling, nipple erection, contraction of the vaginal musculature, and a general tingling sensation.

Specifically, after oral administration of an iodide salt, the female subject experienced increased vaginal lubrication. The term “increased vaginal lubrication,” as used herein, refers to an increased amount of transudate present in the vagina above the amount of transudate present in the vagina above the amount of transudate present in the unstimulated state.

Thus, in one embodiment, the method of increasing vaginal lubrication in a human female described herein, comprises administering to a female in need of said treatment an effective amount of an iodide salt, comprising an iodide salt, water and optionally an acid. The acid can be an organic or an inorganic acid. As described in the Exemplification below, an effective amount of iodide salt for an adult human female weighing approximately 165 pounds, is about a 1.0 to 10M solution, preferably around a 5M solution. It is understood that higher or lower amounts of iodide salt would be used for heavier or lighter subjects as in the purview of one skilled in the art.

It has been discovered that hydriodic acid may be generated in situ by adding water-soluble iodide salts, e.g., alkali metal and alkaline earth metal salts, especially sodium iodide, potassium iodide, magnesium iodide, and the like, to an acidic solution. Suitable such acidic solutions include, e.g., lemon juice, lime juice, other citric acid-containing solutions, vinegar, carbonic acid, phosphoric acid, and the like. It will be appreciated that stomach acid also can serve as the acidic medium for administration of solutions of iodide salts, for generation of hydriodic acid in situ. Flavoring agents may be advantageously added to the solutions of the iodide salts to enhance the flavor and increase the palatability of the solutions. When fruit acids such as lemon juice are used, or when vinegar is used, addition of sugar improves the palatability of the solution.

Thus, as a result of Applicant's discovery, a method is now provided for a neat, hygienic and convenient means of increasing vaginal lubrication in a human female. The invention will be further illustrated by the following non-limiting Exemplification:

### EXAMPLE 1

Hydriodic acid solution was purchased from Eli Lilly and Company, of Indianapolis, Ind., referred to as hydriodic acid syrup. Hydriodic acid syrup can also be prepared by combining hydriodic acid with purified water and dextrose. For example, 140 ml of hydriodic acid is mixed with 550 ml of purified water. To this mixture, 450 g of dextrose is added and dissolved by agitation. Quantity sufficient of purified water is then added to bring the volume to 1000 ml, and the solution is filtered.

The syrup was administered orally to a female human, 52 years of age, weighing approximately 165 pounds. About 0.5 to 3.0 teaspoons of the hydriodic acid syrup was admixed with approximately 8 ounces of tap water and then ingested. An aphrodisiac effect, as defined above, including increased vaginal lubrication, was experienced within approximately 24 hours.

### EXAMPLE 2

To 20 ml of lemon juice are added 3 ml of a 5M solution of sodium iodide in water, and 1 tbsp of sugar is added to the resultant solution. The solution is administered to a female patient, and a reaction similar to that elicited by hydriodic acid syrup in water is observed, including arousal and increased vaginal lubrication.

Equivalents

Those skilled in the art will recognize, or be able to ascertain, using no more than routine experimentation many equivalents to the specific embodiments of the invention described herein. Such equivalents are intended to be encompassed by the following claims.

The invention claimed is:

1. A method of increasing vaginal lubrication in an adult human female in need of said treatment, said treatment consisting essentially of increasing the amount of transudate present in the vagina, comprising orally administering to the female an effective amount of an aqueous solution comprising a pharmaceutically acceptable water soluble iodide salt, wherein hydriodic acid is generated in situ by the stomach acid of said patient.

2. A method of claim 1 wherein the concentration of iodide salt is from about 1M to about 10M.

3. A method of claim 1 wherein the acidic solution further comprises a pharmaceutically acceptable counter ion selected from the group consisting of alkali earth metals or alkaline earth metals.

4. A method of claim 3 wherein the acidic solution further comprises a pharmaceutically acceptable counter ion selected from the group consisting of sodium, potassium, and magnesium.

5. A method of claim 1 wherein the aqueous solution is acidic.

6. A method according to claim 5 wherein the acid aqueous solution comprises lemon juice, lime juice, solutions containing citric acid, vinegar, acetic acid, carbonic acid and phosphoric acid.

7. A method of increasing vaginal lubrication in an adult human female in need of said treatment, said treatment consisting essentially of increasing the amount of transudate present in the vagina, comprising orally administering to the female an effective amount of an aqueous solution of a water soluble iodide salt.
